(19) United States
(12) Patent Application Publication
(10) Pub. No.: US 2006/0211080 A1
(43) Pub. Date: Sep. 21, 2006

(54) Automated biological plate spreader

(75) Inventors: James D. Frost III, The Woodlands, TX (US); Alfred W. Lasher III, Houston, TX (US); Steven G. Widen, Pearland, TX (US)

Correspondence Address: VINSON & ELKINS L.L.P., 1001 FANNIN STREET, 2300 FIRST CITY TOWER, HOUSTON, TX, 77002-6760, US

(73) Assignee: Picoscript Ltd., L.L.P., Houston, TX (US)

(21) Appl. No.: 11/081,277

(22) Filed: Mar. 16, 2005

(51) Int. Cl. C12Q 1/24; C12M 1/36

(52) U.S. Cl. 435/30; 435/286.3; 435/286.4

(57) ABSTRACT

The present invention relates to a robotic apparatus and method for automating the spreading of a biologic sample on a plate, wherein the robot includes at least one effector arm to hold and shake the plate and a pipetting arm to deliver the sample to the plate. The present invention also includes the spreading of the biologic sample being assisted by at least one bead being rolled on the interior bottom surface of the plate and then removed from the plate.

### BACKGROUND OF THE INVENTION

1. Field of the Invention

The present invention utilizes a robotic system to automate the plating of aliquots of samples for biologic assays. Specifically, the present invention relates to apparatus and methods to automate the spreading of micro-organism cultures, top agar or other liquid solutions.

2. Description of the Related Art

Life science experiments and clinical diagnostics often require the use of micro-organisms and/or cell cultures. In such life science applications, the use of these biologics traditionally calls for the spreading or plating of a small quantity of a liquid culture onto the surface of a solid or semi-solid growth medium. These growth mediums, such as agar based media, are well known in the art and often contained in the bottom portion of a Petri dish, or similar type plate. Historically, the plating of such aliquots involves the expulsion of a liquid onto the surface of the growth media followed by the manual spreading of the liquid in a thin film across the entire surface of the media. The liquid aliquot or culture sample is often spread with the aid of a sterilized bacterial loop or glass rod. However, because of the extreme sensitivity of most these applications to contamination (direct or cross), the device used to spread the sample must be manually sterilized between each plating.

Recent advances in the plating of such cultures include the use of multiple glass beads to aid in sample spreading. For example, a practitioner may pipette a sample onto a growth media in a Petri dish, then add several small sterilized glass beads and move the dish in such a fashion as to cause the beads to spread the sample across the media. The beads can then simply be removed and placed into a sterilization solution and a new set of beads used for the next plating. Other advances include the use of spinning turn tables or hand held spreaders to decrease the time involved. Nevertheless, even with these new advances only one sample can be plated at a time and it requires substantial human time, expense and labor to grow these cultures on individual plates.

Furthermore, these biological assays often involve the plating of serial dilutions of samples containing micro-organisms or cells in order to tabulate their concentration or provide a culture with colonies which are dispersed to the extent that an individual colony can be readily isolated. These serial dilutions are often performed in ten fold increases in volume or ten fold reductions in the concentration of the biologic. After the solutions are diluted, they have to be spread on growth medium in order to determine the number of colonies or cells present in the solution. In such assays the spreading of the aliquot on the growth medium in a thin film is imperative, so that the biological units present in the liquid are separated with respect to location, and such assays often require multiple platings per sample thereby increasing the time, expense and labor involved.

Therefore, because the plating of samples on growth media requires human manipulations such as pipetting and spreading of the culture by turning the plate manually, using a turn table, or glass beads, a need exists for an apparatus that is capable of automating the plating of multiple samples. The present invention is directed to overcoming, or at least reducing the effects of, one or more of the problems set forth above.

### SUMMARY OF THE INVENTION

In certain embodiments, the present invention is directed toward a robotic apparatus for automating the spreading of a biologic sample on a plate comprising: a robot, wherein the robot further comprises; at least one effector arm to hold and shake a plate, wherein the plate has an interior bottom surface; and a pipetting arm to deliver the sample to the plate.

In some of these embodiments the robotic apparatus further comprises the automated spreading of the biologic sample being assisted by at least one bead being rolled on the interior bottom surface of the plate. Furthermore, some of these embodiments also comprise an effector to remove the at least one bead from the plate after the at least one effector arm has shaken the plate causing the at least one bead to assist the spreading the biologic sample on the interior bottom surface of the plate. In certain embodiments the effector to remove the at least one bead is a magnetic effector and the at least one bead is magnetic, paramagnetic or superparamagnetic.

In certain other embodiments, the present invention is directed toward a method for the automated plating of a biologic sample comprising using a robotic apparatus to: (1) retrieve a plate having an interior bottom surface; (2) place a sample on the interior bottom surface of the plate or coating thereof; and (3) move the plate to cause the biologic sample to spread across the interior bottom surface of the plate. In some of these embodiments, the method further comprises placing at least one bead on an interior bottom surface of the plate to roll across the interior bottom of the plate during the moving of the plate thereby assisting in the spreading of the biologic sample. In certain of these embodiments the at least one bead is a magnetic, paramagnetic or superparamagnetic bead and is removed from the plate after being rolled on the interior bottom surface by a magnet.

### BRIEF DESCRIPTION OF THE FIGURES

FIG. 1 depicts a schematic of one embodiment of a robotic plate spreader and deck.

FIG. 2 shows one embodiment of an end effector and its associated arms.

FIG. 3 depicts one embodiment of an initial plate receptacle in the form of an agar plate stacker.

FIG. 4 shows a schematic of one embodiment of a bead addition station.

FIG. 5 shows one potential embodiment of a pipette tip removal station.

FIG. 6 depicts one embodiment of a bead removal station.

FIG. 7 shows an infrared sensor.

FIG. 8 represents one embodiment of complete plate receptacle.

### DETAILED DESCRIPTION OF SPECIFIC EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The present invention automates the spreading of microorganism cultures, top agar, viral supernants, and other liquid or semi-liquid samples onto plates using robotics. The apparatus and methods disclosed herein do not require a human to be present while liquid or semi-liquid reagents are spread onto the plates.

Certain embodiments of the apparatus of the present invention include a robot, having at least one robotic arm. The robot can be mounted either directly on a robotic deck or close enough to the deck so as to allow the robotic arm to manipulate items on the robotic deck. The robot in certain embodiments can be controlled and monitored by a Visual Basic-based graphic user interface. The interface can monitor a series of sensors that ensure successful progress of the plating process. The plating process is generally performed by applying a liquid sample and at least one bead to a plate containing a growth media, shaking the beads in the plate using a robotic arm followed by the removal of the beads from the plate. The robot can also be used to carefully and gently pipette viral supernatants and top agar for plaque assays and then gently rotate or shake the plate to allow the liquids to flow across the plate without the use of beads, for example to avoid disturbing a cell monolayer.

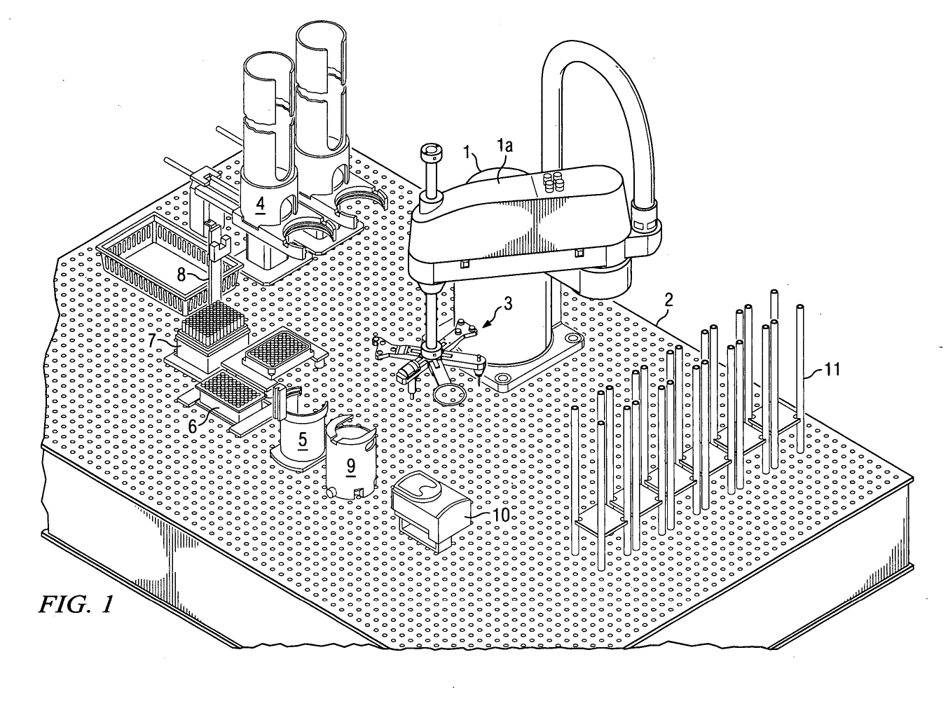

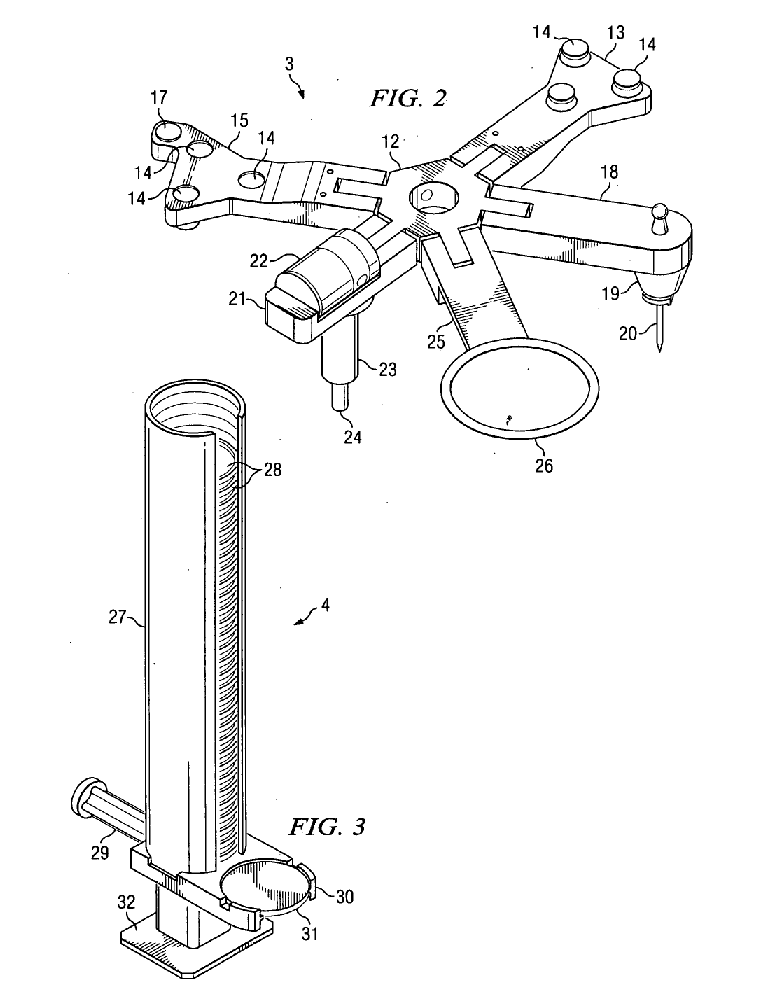

FIG. 1 depicts one embodiment of the present invention which includes a robot 1 having at least one robotic arm 1a that can be mounted on a robotic deck 2. The robotic arm 1a can be attached to an end effector 3 which in turn is attached to several effector arms. In alternate embodiments, the robot 1 may have multiple robotic arms, or multiple end effectors, each one designed to perform a particular function or set of functions and one of ordinary skill in the art will readily recognize that each effector arm, as discussed below, might be utilized in other embodiments as an independent robotic arm 1a or end effector which are therefore within the scope of the present invention. FIG. 2 shows an embodiment of the end effector 3 having multiple effector arms. In some of these embodiments, the end effector 3 includes an end effector base 12 which serves as an attachment point for each effector arm (i.e., the end effector base 12 serves as an attachment between the robotic arm 1a and individual effector arms).

Certain embodiments include a bottom suction arm 13. The bottom suction arm 13 can include one or more suction points 14 on its upper surface. The suction points 14 can be activated to attach to the bottom of a plate through a suction force. In alternate embodiments, the bottom suction arm 13 can have sticking points in place of the suction points 14 to attach to the dish. One of ordinary skill in the art will recognize that multiple mechanisms, including adhesive areas, or claws could be utilized to aid in the attachment of the bottom suction arm 13 to the dish or plate and are therefore within the scope of the present invention.

The end effector base 12 can also be attached to a top suction arm 15. The top suction arm 15 can include suction points 14 on its lower surface to attach to the lid of a plate. As with the bottom suction arm 13, one of ordinary skill in the art will recognize that multiple mechanisms could be utilized to aid in the attachment of the bottom suction arm 13 to the dish or plate. In addition to suction points 14 or alternative attachment points, the top suction arm in certain embodiments also includes a magnetic effector 17. The magnetic effector 17 can create a magnetic field through the plate lid and into the lumen of an enclosed plate.

In some embodiments of the present invention, a magnetic arm 18 is attached to the end effector base 12. The magnetic arm 18, in certain embodiments, includes an elongated spine 20 at its distal most end. In alternate embodiments, the magnetic arm 18 can also include a magnetic force generating unit 19 which when activated magnetizes the elongated spine 20. One of ordinary skill in the art will readily recognize that the magnetic arm 18 can be of multiple suitable configurations that are capable of providing a magnetic force at the arm's distal end.

The end effector base 12 can also be attached to a pipetting arm 21. The pipetting arm 21 of the present invention can facilitate the automated delivery of a liquid or semi-liquid sample to a plate. The pipetting arm 21 may be a pipett, an injector, a syringe or any other structure capable of delivering a liquid or semi-liquid sample. The pipetting arm 21 can include a solution delivery tube 23 having a pipette tip receptacle 24. Certain embodiments of the pipetting arm 21 may contain a solution reservoir 22 to hold a wash or sample fluid. In alternate embodiments the tube may be connected through the robotic arm 1a to an automated solution delivery system, as discussed below, which provides for various solutions to be pipetted through the solution delivery tube 23 in a predetermined fashion. Still other embodiments of the pipetting arm 21 include a pipette tip receptacle 24 connected to a pipetting mechanism that when activated can cause the aspiration or expulsion of a liquid sample for a pipette tip attached to the pipette tip receptacle 24.

Some embodiments of the present invention also include a circular spatula arm 25 connected to the end effector base 12. The circular spatula arm 25 includes a circular ring 26 having an inner circumference sufficient to accommodate the outer circumference of the dish or plate to be used. It would be readily recognized that the circular spatula arm 25 can be of any suitable configuration at its distal end, so long as the configuration matches the size and shape of the plate being used sufficiently to engage and hold the plate.

As shown in FIG. 1, certain embodiments of the present invention can also include an initial plate reservoir 4, a bead addition station 5, a bead reservoir station 6, a pipette tip reservoir 7, a pipette tip removal station 8 and/or a bead removal station 9. Some of these embodiments may also include an infrared sensor 10 for determining the completion of various tasks by the robotic arm. Some embodiments further have a completed plate receptacle 11. All of these stations and items (can be mounted on the robot deck or off the robotic deck 2 but close enough for the robot) to interact with.

In certain embodiments of the present invention, the robot, via a robotic arm 1a or an effector arm, can interact with and retrieve a plate from the initial plate reservoir 4. The initial plate reservoir 4, as shown in FIG. 1, can be mounted on the robotic deck 2 or off the robotic deck 2 but close enough for the robot 1 to retrieve plates from it. The initial plate reservoir 4 can be of any configuration that holds at least one plate and allows the robot to direct the retrieval of a fresh plate for each sample plating. In certain other embodiments, the robot 1 may simply be instructed as to the location of fresh plates without a receptacle, one of ordinary skill in the art will recognize that the present invention can utilize many types of plates but the preferred plate shape is round.

FIG. 3 depicts one embodiment of an initial plate reservoir 4. In this embodiment, the initial plate reservoir 4 includes a plate stacker having a plate reservoir 27 which holds at least one plate. In some of these embodiments, the plates are Petri dishes with an agar layer in the bottom portion. This type of plate is commonly used in biological laboratory practices. In certain embodiments, the plate reservoir 27 may hold up to 48 Petri dishes 28. The initial plate reservoir 4 may also include a plate ejector 29 which serves to eject a single plate at a time into a primary plate holder 31. The initial plate reservoir 4 may further have a base 32 that is capable of being attached to the robotic deck 2.

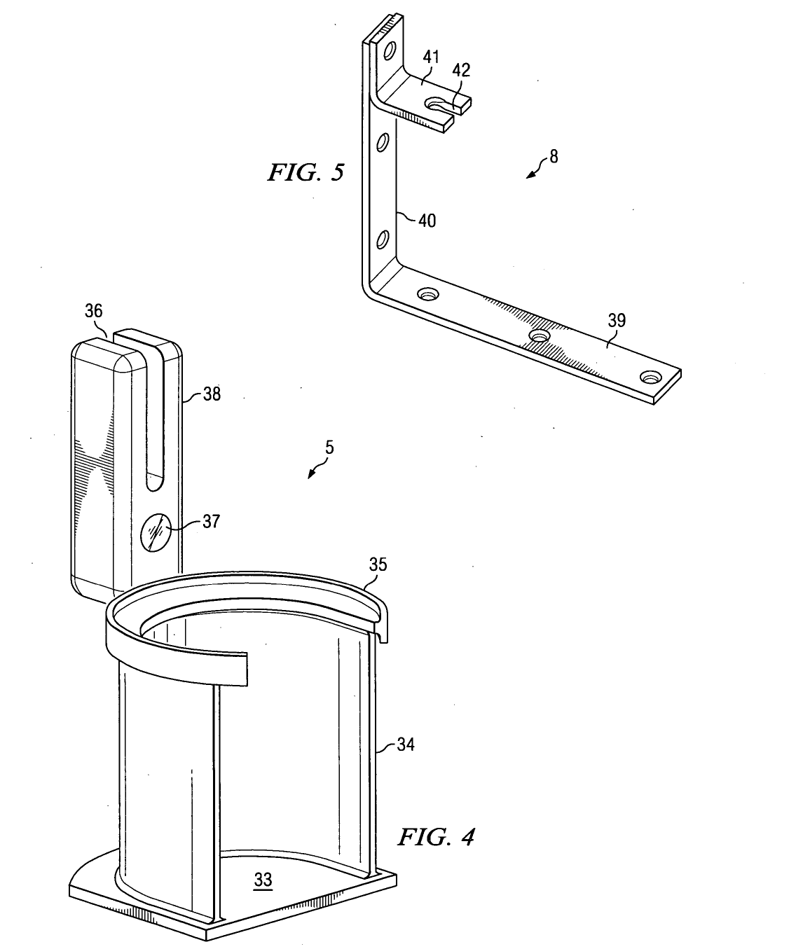

In embodiments of the present invention that include a bead addition station 5, the bead addition station 5 can be at a defined location on the robotic deck 2 where the robot 1 is instructed to add beads to a plate, as later described. For example, in some embodiments, the primary plate holder 31 can also serve as the bead addition station. In certain other embodiments, the bead addition station 5 can be an independent structure of any configuration that is capable of holding the plate being used. FIG. 4 depicts one such embodiment of a bead addition station 5. This particular embodiment includes a base 33, sides 34, and a plate rack 35. The bead addition station 5 further includes a mast 38 extending above the plate rack 35. This mast, in certain embodiments, can include a bar code reader 37 and a bead removal slot 38.

Certain embodiments of the current invention also include an independent pipette tip removal station, while still other embodiments include a tip ejector for the removal of a pipette tip from the pipette tip receptacle 24 on the pipetting arm 21 itself. In certain embodiments that include a separate pipette tip removal station such as shown in FIG. 5, the pipette tip removal station 8 can include a base 39 which may be attached to a robotic deck 2. The base is connected to a vertical extender 40 which in turn is connected to a pipette tip removal bar 41. This pipette tip removing bar contains a notched groove 42 which has a width that is slightly smaller than the outside diameter of the pipette tips to be utilized by a particular given embodiment of this invention. One of ordinary skill in the art will recognize that pipette tips of any size can be readily used in this invention.

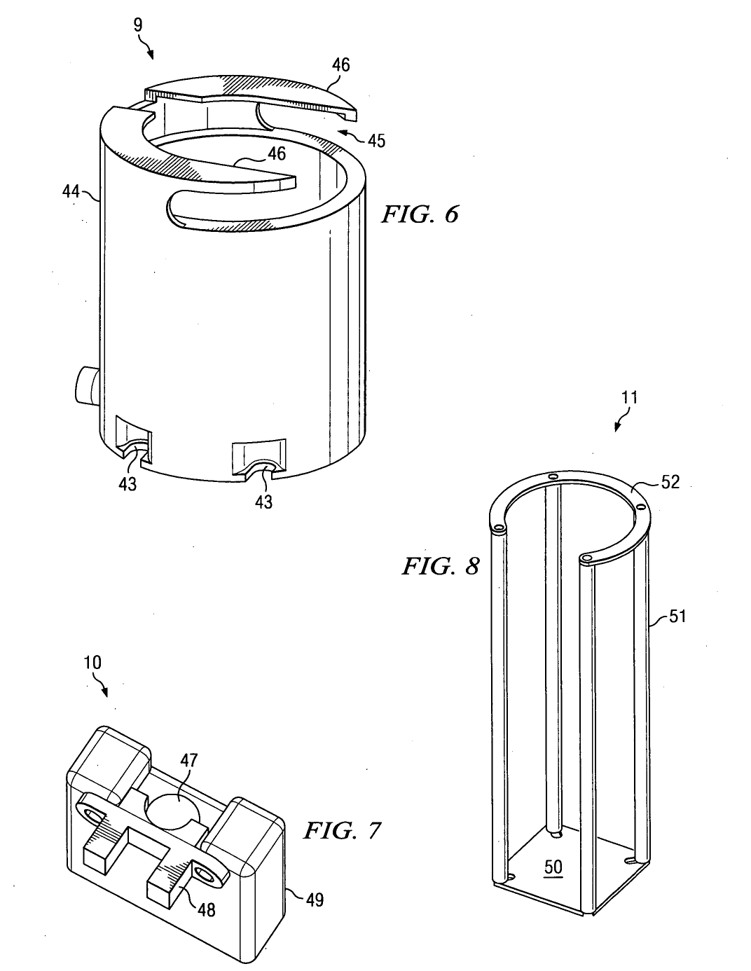

Certain embodiments of the present invention also include a bead removal station 9. The bead removal station 9 may be combined with another station or have an independent physical location. FIG. 6 depicts one embodiment of a bead removal station 9 having a separate independent structure. This particular embodiment contains a base 43 than can be used for attachment to a robotic deck 2, a cylinder 44 for receipt of the beads as they are removed from the plate and a plate lid bracket consisting of upper flanges 46 and a lid notch 45. The lid notch 45 is of a width sufficient to allow the robotic arm 1a or end effector 3 to be manipulated by the robot 1 such that the top suction arm 15 can insert the plate lid into the lid notch, release the lid and have it be held in the lid notch 45.

Certain embodiments of the current invention also include an infrared sensor 10. This infrared sensor 10 can be utilized to verify receipt and removal of a pipette tip from the pipetting arm 21 as well as the receipt, removal and retrieval of beads from the bead addition arm or top suction arm, as described below. In certain embodiments the infrared sensor 10 can contain a base 49, a sensor 47 and a plate bracket 48 as shown in FIG. 7.

In some embodiments of the present invention, a completed plate rack 11 can be used as shown in FIG. 8. Certain embodiments of the completed plate rack 11 include a base 50 for attachment to a robotic deck 2 and receptacle columns 51 for positioning and holding the finished plates. Certain other embodiments of the completed plate rack 11 also include a solid cap 52 to steady and maintain the position of the receptacle columns 51. One of ordinary skill in the art will readily recognize that finished plates (i.e., plates that have had a sample spread on the plate bottom or a growth medium) can be placed in numerous locations. Such locations could include a location as simple as a defined region, a completed plate rack 11, or an incubator shelf.

Certain embodiments of the present invention include an activity verification system. In some of these embodiments, at least one effector arm includes a switch, or other mechanism such as a infrared detector, to inform the robot when a particular task has been accomplished. For example, the top suction arm 15 may include a switch which is activated to inform the robot when the plate lid is properly engaged. Likewise the bottom suction arm 13 and/or the circular spatula arm 25 may contain a switch which is activated to inform the robot when the plate is properly engaged. Furthermore, certain embodiment of the activity verification system include switches or other such mechanisms on stations such as the initial plate reservoir 4, bead addition station 5, bead reservoir station 6, pipette tip reservoir 7, pipette tip removal station 8 and bead removal station 9 to verify for the robot that any particular action as described in this disclosure at such location has been accomplished.

As described above, the robot 1 in certain embodiments can be controlled and monitored through the entire plating process. In some of these embodiments, the plating process is performed by applying beads and a sample to a plate containing a growth media then shaking the plate using a robotic arm, followed by the removal of the beads from the plate. In certain embodiments, a Visual Basic-based graphic user interface is used to operate and monitor the robot, however, one of ordinary skill in the art would readily recognize that multiple operation platforms can be suitably substituted for use in the present invention.

Certain embodiments of the present invention include methods for the automated plating of liquid or semi-liquid samples on a plate. In certain of these embodiments, the bottom of the plate is covered with a growth media of some type such as an agar media. One such method includes the following steps: a robot 1 retrieves a fresh plate and removes the lid; followed by the robot 1 directing the addition of a liquid sample and a set of beads; the robot 1 subsequently directs the moving of the plate to cause to the beads to roll over the interior bottom surface of the plate or growth media, followed by the removal of the beads; the plate is then set aside; and the process repeated with a new plate. In some embodiments, the sample can be added prior to the beads, while in others the beads can be added prior to the sample. In certain embodiments, the beads and the sample are added simultaneously. In some such embodiments, the beads are of sufficiently small diameter to be pipetted along with the sample.

The beads utilized by the current invention may be of any suitable size. In certain embodiments, the beads of the present invention are substantially spherical in shape. In some of these embodiments, the beads are composed, at least in part, of magnetic, paramagnetic or super paramagnetic materials (“magnetic beads”). In certain embodiments, the beads are covered with hydrophilic and/or non-toxic coatings. In such embodiments, the retrieval of beads by the robot 1 can be accomplished through the use of a magnet.

The beads, in still other embodiments, are composed of non-magnetic materials such as plastic or glass. In such embodiments, the robot will physically retrieve the beads through a non-magnetic means such as a scoop, tray, net or similar device. Once delivered to the plate, these beads can be removed by the robot by tilting or inventing the plate and letting the beads fall out, or the robot can direct the active removal of the beads through the use of the scoop, tray, net or similar device.

In certain embodiments, the robot 1 initiates the process by retrieving a single plate from an initial plate receptacle. In some of these embodiments, the retrieval of the plate is initiated by a plate ejector 29 ejecting a plate from the initial plate reservoir 4 into the primary plate holder 31. The plate ejector 29 can be activated by the robot, by a controller interacting with or directed by the robot 1 or may even be automatically ejected into the primary plate holder 31 when it is empty. Following ejection of a plate, the end effector 3 is rotated to extend a bottom suction arm 13 from the robotic arm. The bottom suction arm 13 is then positioned underneath of the ejected plate 31 on the initial plate reservoir 4. The bottom suction arm 13 is then raised to the bottom of the plate and the suction points 14 are activated to attach the bottom suction arm 13 to the ejected plate. In certain embodiments, the bottom suction arm 13 is then lifted and passed over an infrared sensor 10 to verify that a plate is attached. If the infrared sensor 10 does not detect a plate, the process is reinitiated by returning the bottom suction arm 13 to the initial plate receptacle. Once the infrared sensor 10 detects a plate is present on the bottom suction arm, the robotic arm 1a repositions the bottom suction arm 13 to place the plate into a bead addition station. In certain embodiments, the positioning of a plate in the bead addition station 5 activates a bar code reader 37 to record a bar code identification on a given plate and record the identity of a specific plate with respect to the plating of a particular sample.

After a plate is placed on the bead addition station, the robotic arm 1a lowers the bottom suction arm 13 and the suction points 14 are inactivated to release the bottom suction arm 13 from the plate, leaving the plate in the bead addition station. The bottom suction arm 13 is then retracted away from the bead addition station.

The end effector 3 is then rotated to allow the robot to use the magnet arm to engage a bead reservoir station. In certain embodiments, the bead receptacle is a 96-well plate holding beads in one or more wells. In such embodiments, the robot 1 can be instructed to retrieve beads from a specific well for each individual plating. In alternative embodiments, the bead reservoir station consists of a single bead receptacle which holds beads for multiple platings. In embodiments using magnetic beads, the magnetic arm is then activated to retrieve beads from the bead reservoir. In certain embodiments of the magnetic arm (such as shown in FIG. 2), the magnetic arm includes an elongated spine 20 can be lowered into the bead reservoir station 6 by the robot 1 to allow the magnet to collect beads. In embodiments that include sets of beads in certain predefined locations, such as in a well of a 96-well plate, the elongated spine 20 can be lowered into the predefined location (or well) and activated to retrieve beads from such location. The magnetic arm is then removed from the bead reservoir. In alternate embodiments, the magnetic arm is always magnetic and does not need to be activated. In certain embodiments, the magnetic arm is then passed over an infrared sensor 10 to verify the presence of the beads on the magnetic arm. If beads are not detected by the infrared sensor, the magnetic arm is repositioned to engage the bead reservoir and retrieval process is reinitiated. In embodiments utilizing non-magnetic beads, the robotic effector arm can be a scoop, tray, net or similar structure to physically retrieve the beads.

After beads have been retrieved from the bead reservoir by the magnetic arm or on-magnetic effector arm, the robot retracts the arm (from the infrared sensor 10 in embodiments with an infrared sensor) and rotates the end effector 3 to allow the robot 1 to position the pipetting arm 21 over the pipette tip reservoir 7. The pipetting arm 21 is then lowered into a predefined location within the pipette tip reservoir 7 causing the pipette tip receptacle 24 of the pipetting arm 21 to engage a single pipette tip. In certain embodiments, the pipette tip reservoir 7 is a common 96-tip box of pipette tips. In these embodiments, the robotic arm 1a is instructed to retrieve a tip from a specified location within the box. Once the pipette tip has been engaged by the pipette tip receptacle 24 of the pipetting arm 21 it raises away from the pipette tip reservoir. In certain embodiments, the pipetting arm 21 is passed over an infrared sensor to verify that one and only one tip was removed from the pipette tip reservoir. If the infrared sensor 10 does not detect the presence of a single pipette tip, the robotic arm 1a will be returned to the pipette tip reservoir 7 for retrieval of a pipette tip or directed to eject the tips as described below and returned to the pipette tip reservoir.

After retrieving a pipette tip from the pipette tip reservoir, the robotic arm 1a positions the pipetting arm 21 over a sample source plate 6. The sample source plate 6 contains the samples that will be spread onto the plate(s). The pipetting arm 21 is then lowered to place the pipette tip into the sample source plate 6 and a sample is aspirated into the pipette tip. In certain embodiments, the sample source plate 6 is a 96-well plate and the robotic arm 1a is instructed to retrieve a sample from a specified location/well within the 96-well plate for each individual plating. Following the aspiration of the sample, the robotic arm 1a will raise the pipetting arm 21 from the sample source plate 6 and rotate the end effector 3 to allow the robot 1 to position the top suction arm 15 over the bead addition station. One of ordinary skill in the art would readily recognize that a wide variety of sample source plate formats will function in and are within the scope of the present invention. For example, individual conical tubes can be used in the place of the 96-well plate.

In alternative embodiments were the same sample is to be spread or loaded onto multiple plates, the pipetting arm 21 can be attached to a sample reservoir 22. In such embodiments the retrieval of a new pipette tip between each plating may be omitted. In still other embodiments, the pipetting arm 21 can be attached to a sample delivery system through a solution delivery tube. In these embodiments, the sample delivery system can be programmed to deliver a set quantity of sample or diluted sample to each specific/subsequent plate. For example the sample delivery system can be programmed to automate serial dilutions from a particular sample. In these instances, the steps related to retrieval of a sample from the sample source plate may also be omitted.

After extending the top suction arm 15 and positioning it above the plate in the bead addition station, the robotic arm 1a then lowers the top suction arm 15 to engage the lid of the plate and the suction points 14 are activated. The robotic arm 1a then lifts the top suction arm 15 to remove the lid from the plate in the bead addition station. In certain embodiments, an infrared sensor 10 can then be used to confirm that the plate lid is attached to the top suction arm. Following the removal of the plate lid, in embodiments utilizing magnetic beads, the end effector 3 is rotated to allow the robot 1 to position the magnetic arm, including the beads, over the plate in the bead addition station. In certain embodiments, the magnetic arm then releases the beads into the release station cavity and the beads slide onto the plate bottom or growth media. In certain other embodiments that include an elongated spine, the elongated spine 20 of the magnetic arm is pushed through a bead removal slot 38 by the robotic arm. This action physically forces the beads off of the elongated spine 20 and into the release station cavity where they slide onto the plate bottom or growth media.

In embodiments utilizing non-magnetic beads the arm is rotated to a position over the plate and then maneuver the arm to dump or deliver the beads onto the plate.

Once the beads have been delivered to the plate, the end effector 3 can then be rotated to allow the robot 1 to position the pipetting arm 21 over the plate where the pipetting arm 21 can dispense the sample into the plate. After delivery of the sample, the end effector 3 then rotates to reposition the top suction arm 1S over the plate and the robotic arm 1a lowers the top suction arm 15 to replaces the lid on the plate. The top suction arm's suction points 14 are then deactivated and the top suction arm 15 is raised, leaving the lid on the plate.

In alternate embodiments, the use of the beads can be omitted as one of ordinary skill in the art would readily recognize that such steps might be detrimental to the plating process under certain circumstances. For example, in platings of fluids such as using viral supernatants or top agar, or in cases where a cell monolayer is present on the plate, all steps involving the use of beads may be omitted. The robot 1 can carefully and gently pipette viral supernatants and top agar for plaque assays and allow the liquids to flow across the plates to avoid any unnecessary perturbation of the sample, growth media, or cell mono-layer.

After the top suction arm 15 is raised from the plate, the end effector 3 is rotated to allow the robot 1 to position the pipetting arm 21 at the pipette tip removal station. The pipetting arm 21 is positioned such that the pipette tip removal bar 41 is above the proximal end of the pipette tip with the pipette tip receptacle 24 in the notched groove 42 of the pipette tip removal bar. The robotic arm 1a then raises the pipetting arm 21 causing the pipette tip removal bar 41 to force the pipette tip off of the distal end of the pipette tip receptacle. In alternate embodiments, the pipetting arm 21 includes a pipette tip ejector adjacent to the pipette tip receptacle 24 that ejects the pipette tip from the pipette tip receptacle 24 upon activation. In some embodiments, the pipetting arm 21 is passed over an infrared sensor 10 to verify the removal of the pipette tip.

Following removal of the pipette tip, the end effector 3 is rotated to allow the robot 1 to position the bottom suction arm 13 under the plate for removal from the bead addition station. The robotic arm 1a then raises the bottom suction arm 13 to the bottom of the plate with the suction points 14 activated. The bottom suction arm 13 is then raised to remove the plate from the bead addition station. The robotic arm 1a then moves the bottom suction arm 13 to a position that is clear of obstructions and moves the plate in a rapid shuffling pattern thereby rolling the beads about the plate in a fashion that spreads the sample over the surface of the plate bottom or growth media. One of ordinary skill in the art will readily recognize that the movement of the plate by the robot 1 maybe of any pattern or even random depending upon the time period the robot 1 moves the plate and the number of beads present; provided, however, that the beads are rolled across a substantial portion (for example, in some embodiments greater than 50%) of the plate bottom or growth media or partition thereof.

Following the spreading of the sample, the robotic arm 1a redirects the plate to the bead addition station. The suction points 14 are inactivated and the bottom suction arm 13 lowered thus releasing the plate and leaving it on the bead addition station. The robotic arm 1a retracts the bottom suction arm 13 and rotates the end effector 3 to allow the robot 1 to position the top suction arm 15 over the center of the plate. The suction points 14 of the top suction arm 15 are activated and the top suction arm 15 is lowered onto the plate. The top suction arm 15 is then raised, carrying the plate lid. In certain embodiments utilizing magnetic beads, the top suction arm 15 includes a magnetic effector 17 which is constantly magnetized causing the beads to adhere to the plate lid during removal. Still other embodiments include an activatable magnetic effector 17 that is activated while the top suction arm 15 is attaching to the plate lid and when activated causes the beads to adhere to the plate lid. In certain embodiments, an infrared sensor 10 is used to confirm that the plate lid was removed by the top suction arm 15. The robotic arm 1a then repositions the top suction arm 15 so that it places the plate lid and beads at the bead removal station. The lid is placed in the lid notch 45 and under the upper flanges 46 of the bead removal station. The suction points 14 of the top suction arm 15 are then deactivated and the robotic arm 1a lifts the top suction arm 15 away from the lid to a distance sufficient to break the magnetic contact of the top suction arm's magnetic effector 17 with the beads under the plate lid thereby releasing the beads into the bead removal station. The suction points 14 of the top suction arm 15 are then activated and the robotic arm 1a repositions the top suction arm 15 onto the lid in the bead removal station. The lid is then removed from the bead removal station 9 and returned to the plate in the bead addition station. In embodiments having an activatable magnetic effector, the magnet can be deactivated to release the beads into the bead removal station 9 without the need for inactivating the suction points 14 of the top suction arm 15 (in other words the top suction arm 15 remains attached to the lid during bead removal). After the lid is placed on the plate in the bead addition station, the top suction arm 15 suction points 14 are inactivated, leaving the lid on the plate as the robotic arm 1a raises the top suction arm 15 away from the bead addition station.

In embodiments utilizing non-magnetic beads, the effector arm will retrieve the plate after removal of the lid and invert the plate to remove the beads. In some of these embodiments, the effector arm performing this task is the circular spatula arm.

After the top suction arm 15 is lifted above the bead addition station, the end effector 3 is rotated to allow the robot 1 to position the circular spatula arm 25 under the plate at the bead addition station. The robotic arm 1a then lifts the plate from the bead addition station 5 and, in some embodiments, moves it to a barcode station. The circular spatula arm 25 holds the plate over a barcode reader in order to record what sample was placed onto a particular plate. The use of the barcode reader requires the plates have barcodes prior to operating the robot. Following the reading of the barcode, the robotic arm 1a directs the circular spatula arm 25 to a completed plate rack 11. In certain embodiments there are multiple finished plate racks. For example, in one such embodiment there are six finished plate racks, wherein each rack can hold up to 16 Petri dishes. The circular spatula arm 25 places the plate containing the spread sample into a completed plate rack 11. In certain embodiments, the receptacle columns 51 are positioned in such a manner as to be separated by a distance which is more than the diameter of the circular ring 26 of the circular spatula arm 25 but less than the diameter of the plate. In this configuration, the receptacle columns will remove and retain the plate in the completed plate rack 11 when the circular spatula arm 25 is retracted.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below

What Is Claimed:

1. A robotic apparatus for automating the spreading of a biologic sample on a plate comprising:
a robot, wherein the robot further comprises;
at least one effector arm to hold and shake a plate, wherein the plate has an interior bottom surface; and
a pipetting arm to deliver a sample to the plate.

2. The robotic apparatus of claim 1, wherein the spreading of the biologic sample is assisted by at least one bead being rolled on the interior bottom surface of the plate.

3. The robotic apparatus of claim 2, further comprising an effector to remove the at least one bead from the plate after the at least one effector arm has shaken the plate causing the at least one bead to assist the spreading the biologic sample on the interior bottom surface of the plate.

4. The apparatus of claim 3, wherein the effector to remove the at least one bead is a magnetic effector and the at least one bead is magnetic, paramagnetic or superparamagnetic.

5. The apparatus of claim 2, wherein the robot further comprises a top suction arm to remove and replace a lid from the plate by attaching to the lid through a suction force.

6. The apparatus of claim 5, further comprising a magnetic effector to remove the at least one bead on the top suction arm.

7. The apparatus of claim 1, wherein the robot further comprises a circular spatula arm.

8. The apparatus of claim 1, wherein the at least one effector arm is a bottom suction arm that attaches to the plate through a suction force.

9. The apparatus of claim 1, further comprising a robotic deck supporting the robot.

10. The apparatus of claim 9, further comprising a bead addition station attached to the robotic deck.

11. The apparatus of claim 10, further comprising an initial plate receptacle, a bead removal station, a pipette tip reservoir, a completed plate receptacle, a bar-code reader and an infrared sensor attached to the robotic deck.

12. The apparatus of claim 9, further comprising at least one of the following: a bead addition station, an initial plate receptacle, a bead removal station, a pipette tip reservoir, a completed plate receptacle, a bar-code reader or an infrared sensor attached to the robotic deck.

13. The apparatus of claim 1, wherein the interior bottom surface of the plate comprises a solid or semi-solid growth media.

14. A method for the automated plating of a biologic sample comprising using a robotic apparatus to:
retrieve a plate having an interior bottom surface;
place a sample on the interior bottom surface of the plate or coating thereof; and
move the plate to cause the biologic sample to spread across the interior bottom surface of the plate.

15. The method of claim 14, wherein the interior bottom surface of the plate comprises a solid or semi-solid growth media.

16. The method of claim 15, further comprising placing at least one bead on an interior bottom surface of the plate to roll across the interior bottom of the plate during the moving of the plate thereby assisting in the spreading of the biologic sample.

17. The method of claim 16, wherein the at least one bead is a magnetic, paramagnetic or superparamagnetic bead.

18. The method of claim 17, wherein the at least one bead is removed from the plate after being rolled on the interior bottom surface or coating thereof by a magnet.

19. The method of claim 15 wherein the biologic sample is spread over at least 50% of the interior bottom surface of the plate or coating thereof.

20. A method for the automated spreading of a biologic sample in a plate comprising using a robot to:
retrieve a plate having an internal bottom surface from an initial plate receptacle;
move the plate to a bead addition station;
retrieve at least one bead from a bead reservoir station;
retrieve a sample from a sample source plate;
deliver the biologic sample to the internal bottom surface of the plate;
deliver the at least on bead to the internal bottom surface of the plate;
move the plate to cause the at least one bead to roll on the internal bottom surface of the plate thereby spreading the biologic sample on the internal bottom surface of the plate; and
remove the bead from the plate.

21. The method of claim 20, further comprising the robot placing a lid on the plate.

22. The method of claim 21, wherein the robot places the plate into an incubator after returning the lid to the plate.

23. The method of claim 20, wherein the at least one bead is a magnetic, paramagnetic or superparamagnetic bead.

24. The method of claim 23, wherein the at least one bead is retrieved and removed by a magnetic effector.

25. The method of claim 20, wherein the lid is removed from the plate by an effector arm of the robot using a suction force.

26. The method of claim 20, wherein the biologic sample is spread over at least 50% of the internal bottom surface of the plate.
